(19) United States
(12) Patent Application Publication
(10) Pub. No.: US 2025/0152292 A1
(43) Pub. Date: May 15, 2025

(54) Modular Foreskin Restoration Device with Spring-Loaded Tensioning Mechanism

(71) Applicant: Tamir Levy Lomholt, Zichron Ya'acov (IL)

(72) Inventor: Tamir Levy Lomholt, Zichron Ya'acov (IL)

(21) Appl. No.: 19/007,618

(22) Filed: Jan. 2, 2025

Related U.S. Application Data
(63) Continuation-in-part of application No. 17/981,503, filed on Nov. 7, 2022.

(51) Int. Cl. A61B 90/00

(52) U.S. Cl. CPC A61B 90/02

(57) ABSTRACT

A modular foreskin restoration device comprising a first skin-contacting assembly for securing the foreskin, a second skin-contacting assembly positioned in spaced relation to engage the glans, and a tensioning assembly disposed between them. The tensioning assembly includes a first modular coupling element, a second modular coupling element, and at least one spring element positioned between the coupling elements to generate adjustable force along an axis. A retention mechanism, positioned above the first skin-contacting assembly, comprises a body portion with openings for threading elastic cords and a central opening for alignment along the axis. The device further includes modular coupling means enabling detachable and interchangeable connections between the components, allowing customization of the configuration. The first skin-contacting assembly is adjustable in tension and orientation via elastic cords, and the first and second skin-contacting assemblies are configured for angular and positional adjustment relative to the axis, accommodating variations in user anatomy.

### FIELD OF INVENTION

The present invention relates generally to foreskin restoration devices. More specifically, the present invention relates to a modular foreskin restoration device configured to apply adjustable levels of dynamic, spring-loaded tension to the foreskin of a user, while offering modular components that provide flexibility in configuration and allow the device to accommodate different body shapes, user preferences, and varying degrees of foreskin length and tautness.

### BACKGROUND

Although the idea of restoring foreskin may seem unusual to some, there are numerous valid reasons for pursuing it. The foreskin of intact males plays a role in producing pheromones, which can increase a man's attractiveness to others.

Removal of the foreskin also eliminates its natural gliding and “lubricating” function. Studies suggest that, among women who express a preference, the majority favor intercourse with intact men, and they are significantly more likely to experience multiple orgasms with them. Additionally, circumcision reduces male sexual pleasure due to the loss of millions of sensory nerve endings and the transformation of the glans into a rough, cornified surface with diminished sensitivity.

These considerations have led many men to pursue foreskin restoration. However, current methods for foreskin restoration remain limited.

Surgical approaches involve creating a new foreskin by grafting donor skin, typically from the scrotum, onto the penis. This procedure is costly and carries the risks inherent in surgery, including scarring, infection, and complications from anesthesia. The resulting foreskin lacks the smoothness, sensation, and gliding action of natural foreskin. Additionally, recovery from the surgery is lengthy and prevents sexual activity during healing.

Restoration through non-surgical methods is widely recommended by organizations focused on foreskin restoration. By applying tension to the skin, these methods encourage the natural growth of new tissue.

Non-surgical methods avoid the disadvantages of surgery and can produce results that closely resemble a natural foreskin in both appearance and function. However, they are time-consuming, often requiring several years to achieve significant results, and current devices have significant limitations.

Existing foreskin restoration devices generally employ four primary methods of applying tension to the skin, each with drawbacks:
Method 1: Air Pressure Devices: Devices that inflate the foreskin with air to create tension suffer from air leakage, requiring frequent reapplication. They also necessitate the use of tape to prevent air from entering the urethra, which can be cumbersome and uncomfortable.
Method 2: Weighted Devices: These devices use weights to stretch the foreskin. While effective, they are bulky, uncomfortable for prolonged use, and visible through clothing, limiting discretion.
Method 3: Elastic Strap Devices: Devices using elastic straps, which attach to various parts of the body, restrict movement and are often visible when wearing shorts or light clothing. Additionally, the tension applied varies significantly with changes in body position, making it difficult to achieve consistent stretching.
Method 4: Bi-Directional Tension Devices: These devices apply tension through a combination of a pusher that presses the glans toward the body and a pulling mechanism to stretch the foreskin. While offering better control, current designs rely on rigid rods, elastic bands, or fixed springs, each with limitations. Elastic bands are hard to adjust precisely, lock screws create non-dynamic tension that fluctuates with body position, and fixed springs lack adaptability.

Another common drawback of existing devices is the lack of freedom of movement in skin-contacting elements, which are typically constrained along the axial direction of a central rod. This rigidity fails to accommodate variations in foreskin length, tautness, and glans shape, leading to uneven tension and suboptimal results.

Furthermore, existing devices are often singular, fixed systems that do not allow users to adapt configurations to their specific needs or anatomical differences.

It is within this context that the present invention is provided, introducing a modular system with spring-loaded tensioning to address these challenges while improving user comfort, adaptability, and effectiveness.

### SUMMARY

The present invention provides a modular foreskin restoration device designed to apply dynamic tension to the foreskin using a spring-loaded tensioning mechanism. The device comprises a first skin-contacting assembly for securing the foreskin, a second skin-contacting assembly positioned in spaced relation to the first assembly to engage the glans, and a tensioning assembly disposed between the two. The tensioning assembly includes a first modular coupling element, a second modular coupling element, at least one spring element positioned between the coupling elements for generating adjustable force, and a retention mechanism positioned above the first skin-contacting assembly. Modular coupling means connect the components, allowing interchangeability, customization, and replacement of parts.

The invention enables angular and positional adjustment of the first and second skin-contacting assemblies relative to the axis of the device, accommodating variations in user anatomy and ensuring even distribution of tension. Elastic cords threaded through the retention mechanism and the top of the first skin-contacting assembly provide additional tension and orientation control. The modular construction of the device allows users to adapt the configuration to suit individual needs, such as varying the tension, selecting alternative components, or simplifying the structure with fixed spacers.

In some embodiments, the first skin-contacting assembly comprises inner and outer concentric gripping elements made from flexible material. This design enhances securement of the foreskin and ensures adaptability to varying skin lengths and tautness.

In further embodiments, the tensioning assembly includes a second spring element, allowing a combination of two springs to provide increased tension and enhanced stability during use.

In yet further embodiments, the spring elements may vary in diameter, stiffness, or length, providing users with a range of configurations to match their preferences and ensuring precise control over the applied tension.

In some embodiments, the retention mechanism comprises a central threaded opening that allows adjustment of its axial position relative to the first skin-contacting assembly.

In further embodiments, the retention mechanism may include a threaded rod for axial adjustments or a fixed spacer element for a simplified configuration, both of which allow control over the distance between the retention mechanism and the first skin-contacting assembly.

In yet further embodiments, the modular coupling means include interlocking fasteners such as threaded connectors, snap-fit connectors, or bayonet connectors, facilitating secure and detachable assembly of the device components.

In some embodiments, the elastic cords are threaded through openings in the retention mechanism and the top of the first skin-contacting assembly, allowing adjustment of their length to modify the tension applied to the foreskin and the orientation of the first skin-contacting assembly.

In further embodiments, the second skin-contacting assembly includes a concave surface configured to conform to the glans, ensuring secure and comfortable engagement during use.

In yet further embodiments, the housing of the tensioning assembly is configured to accommodate springs of different lengths, offering additional customization options for tension force and overall device configuration.

In some embodiments, the first skin-contacting assembly includes a foreskin alignment ridge to assist in positioning and securing the foreskin.

In further embodiments, the second skin-contacting assembly includes an air pad adjacent to the contact surface, providing cushioning to minimize pressure on the glans.

In yet further embodiments, the device is compact and discreet, allowing it to be worn under clothing with minimal visibility.

In some embodiments, the first skin-contacting assembly may be detachably coupled to the retention mechanism using Velcro, friction fit, or adhesive means, enabling ease of assembly and replacement of components.

The invention provides a retention mechanism with a plurality of openings distributed radially about its outer circumference to enable threading of elastic cords, which are adjustable in length. These cords are configured to provide customizable tension and orientation adjustment of the first skin-contacting assembly relative to the retention mechanism.

The modular design ensures flexibility, adjustability, and customization to accommodate a wide range of user preferences and anatomical variations. The ability to combine, replace, or modify components allows the device to provide effective and versatile tensioning, ensuring comfort and usability for foreskin restoration.

### BRIEF DESCRIPTION OF THE DRAWINGS

Various embodiments of the invention are disclosed in the following detailed description and accompanying drawings.

Common reference numerals are used throughout the FIGURES and the detailed description to indicate like elements. One skilled in the art will readily recognize that the above FIGURES are examples and that other architectures, modes of operation, orders of operation, and elements/functions can be provided and implemented without departing from the characteristics and features of the invention, as set forth in the claims.

### DETAILED DESCRIPTION AND PREFERRED EMBODIMENT

The following is a detailed description of exemplary embodiments to illustrate the principles of the invention. The embodiments are provided to illustrate aspects of the invention, but the invention is not limited to any embodiment. The scope of the invention encompasses numerous alternatives, modifications and equivalent; it is limited only by the claims.

Numerous specific details are set forth in the following description in order to provide a thorough understanding of the invention. However, the invention may be practiced according to the claims without some or all of these specific details. For the purpose of clarity, technical material that is known in the technical fields related to the invention has not been described in detail so that the invention is not unnecessarily obscured.

### Definitions

The terminology used herein is for the purpose of describing particular embodiments only and is not intended to be limiting of the invention.

As used herein, the term “and/or” includes any combinations of one or more of the associated listed items.

As used herein, the singular forms “a,” “an,” and “the” are intended to include the plural forms as well as the singular forms, unless the context clearly indicates otherwise.

It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

When a feature or element is described as being “on” or “directly on” another feature or element, there may or may not be intervening features or elements present. Similarly, when a feature or element is described as being “connected,” “attached,” or “coupled” to another feature or element, there may or may not be intervening features or elements present. The features and elements described with respect to one embodiment can be applied to other embodiments.

The use of spatial terms, such as “under,” “below,” “lower,” “over,” “upper,” etc., is used for ease of explanation to describe the relationship between elements when the apparatus is in its proper orientation.

The terms “first,” “second,” and the like are used to distinguish different elements or features, but these elements or features should not be limited by these terms. A first element or feature described can be referred to as a second element or feature and vice versa without departing from the teachings of the present disclosure.

The term “tensioning assembly” refers to any mechanism or combination of components capable of applying a controlled force between two assemblies. This includes, but is not limited to, spring-based mechanisms, elastic bands, and hydraulic or pneumatic tensioners. In one example implementation, the tensioning assembly comprises at least one compression spring positioned between a first and a second modular coupling element, operable to generate an adjustable force along an axis between the assemblies.

The term “modular coupling means” refers to any structural or mechanical interface that allows detachable and interchangeable connections between components. This includes, but is not limited to, threaded connectors, snap-fit interfaces, bayonet connectors, Velcro, and friction fits. In one example implementation, the modular coupling means involves a threaded connector engaging with the retention mechanism and the first skin-contacting assembly to provide a secure but removable connection.

The term “skin-contacting assembly” refers to any structure designed to engage with the user's foreskin or glans. This includes, but is not limited to, gripping elements, pads, and silicone layers. In one example implementation, the first skin-contacting assembly comprises concentric inner and outer gripping elements made from flexible materials, such as medical-grade silicone, configured to securely hold the foreskin. The second skin-contacting assembly includes a concave surface to comfortably engage the glans.

The term “spring element” refers to any elastic component capable of storing and releasing mechanical energy. This includes, but is not limited to, compression springs, extension springs, and torsion springs. In one example implementation, the spring element is a helical compression spring made of stainless steel, chosen for its durability, resistance to deformation, and ability to maintain consistent force during use.

The term “retention mechanism” refers to any structure or system that maintains the position of a component within a defined range or allows adjustment of component positions. This includes, but is not limited to, threaded connections, locking pins, and ratchet mechanisms. In one example implementation, the retention mechanism comprises a body portion with openings distributed radially about its outer circumference for threading elastic cords, and a central threaded opening to facilitate axial adjustments.

The term “elastic cords” refers to any flexible, stretchable material capable of transferring tension or adjusting the orientation of components. This includes, but is not limited to, elastomeric polymers, rubber, or silicone materials. In one example implementation, the elastic cords are threaded through openings in the retention mechanism and the top of the first skin-contacting assembly, enabling adjustment of length and tension to modify the orientation and force applied to the foreskin.

The term “spacer element” refers to any component that increases the separation distance between assemblies or replaces adjustable components such as threaded rods. This includes, but is not limited to, rigid inserts, adjustable sliders, or telescoping segments. In one example implementation, the spacer element is a bioplastic insert of fixed length that connects the retention mechanism to the first skin-contacting assembly, maintaining a consistent axial distance.

### DESCRIPTION OF DRAWINGS

The present invention provides a modular foreskin restoration device that addresses the limitations of prior art by incorporating a spring-loaded tensioning mechanism and a modular construction. Traditional devices, as discussed in the background, suffer from limited adaptability, lack of dynamic tension control, and user discomfort during extended use. Many existing designs rely on rigid rods or fixed tension mechanisms, which fail to accommodate variations in anatomy and movement, resulting in uneven tension distribution and suboptimal outcomes.

The modular design of the present invention allows users to tailor the device configuration to meet their individual needs. The device comprises a first skin-contacting assembly for securing the foreskin, a second skin-contacting assembly for engaging the glans, and a tensioning assembly disposed between the two to apply controlled, adjustable force. The tensioning assembly includes at least one spring element positioned between modular coupling elements, enabling the application of dynamic tension that adjusts to user movement while maintaining consistent stretching. The modular coupling means allow for the easy attachment and interchangeability of components, facilitating customization, component replacement, or modifications to the tensioning system as desired.

Unlike prior designs that lack flexibility, the present invention offers angular and positional adjustability of the skin-contacting assemblies relative to the axis of the device. This adjustability ensures even tension application across the foreskin, accommodating anatomical differences such as variations in skin length and tautness. The inclusion of elastic cords, which are adjustable in length and threaded through a retention mechanism, further enhances the precision of tension application and orientation control.

The modular coupling means provide further versatility, enabling the integration of alternative components, such as springs of different stiffnesses or lengths, spacers for fixed configurations, or alternative gripping elements. This flexibility allows the user to adapt the device to their needs, whether for beginners or advanced users requiring higher tension or specialized configurations.

By combining dynamic tension control, modularity, and ergonomic design, the invention provides a compact, discreet, and user-friendly solution for foreskin restoration. Its design ensures adaptability to a wide range of user preferences and anatomical variations while maintaining consistent and effective operation.

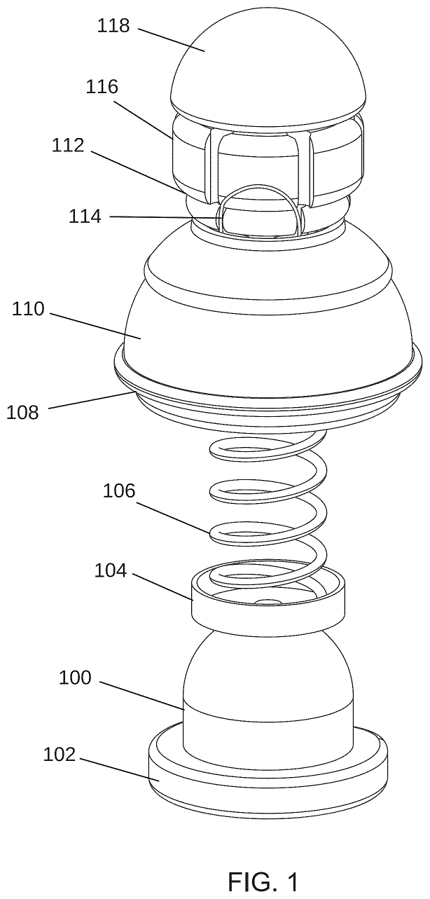

FIG. 1 illustrates the assembled configuration of the modular foreskin restoration device in its uncompressed state. The device includes a second skin-contacting assembly 100 at the bottom, configured as a concave pusher element with an outer rim 102. This component is designed to engage with the glans of a user and may, in some embodiments, incorporate an air pad for additional comfort and cushioning. The second skin-contacting assembly 100 may be constructed from materials such as medical-grade silicone or other biocompatible materials to ensure safe and prolonged contact with the skin. The base of the assembly includes a first modular coupling element 104 for connecting to the spring 106.

The spring 106, shown in its fully extended configuration, serves as the central tensioning element of the device. In this example, the spring 106 is a helical compression spring, which may vary in stiffness or length depending on the desired tensioning force. The spring 106 connects the second skin-contacting assembly 100 to the first skin-contacting assembly 108 through modular coupling elements. These coupling elements allow for the secure yet detachable integration of the spring 106 and other components, enabling users to replace or customize the spring 106 as needed. Alternative spring configurations, such as extension springs or dual-spring combinations, may also be employed to provide adjustable or enhanced tensioning.

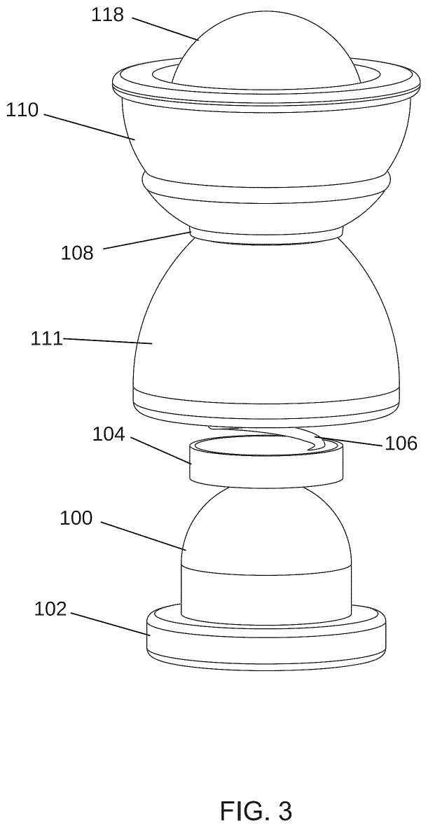

The first skin-contacting assembly 108 is positioned above the spring 106 and includes an outer gripping element 110 and an inner gripping element (not visible in FIG. 1). The outer gripping element 110 is dome-shaped and secures the foreskin during use. This component, like the second skin-contacting assembly 100, may be constructed from flexible and biocompatible materials such as silicone. The outer gripping element 110 may be inverted and raised, as shown in FIG. 3, to reveal the inner gripping element, which assists in securing the foreskin.

Above the first skin-contacting assembly 108 is the retention mechanism 112. The retention mechanism 112 comprises a structure with a series of grooves/openings distributed around its circumference for threading elastic cords 114, which are partially visible in FIG. 1. These elastic cords 114 are adjustable in length and help to modify the orientation and tension of the first skin-contacting assembly 108 relative to the axis of the device. In some embodiments, the retention mechanism 112 may include a central threaded opening for engaging with a threaded rod 118 (only cap visible in FIG. 1), allowing axial adjustments to the distance between the retention mechanism 112 and the first skin-contacting assembly 108 by turning an adjuster knob 116.

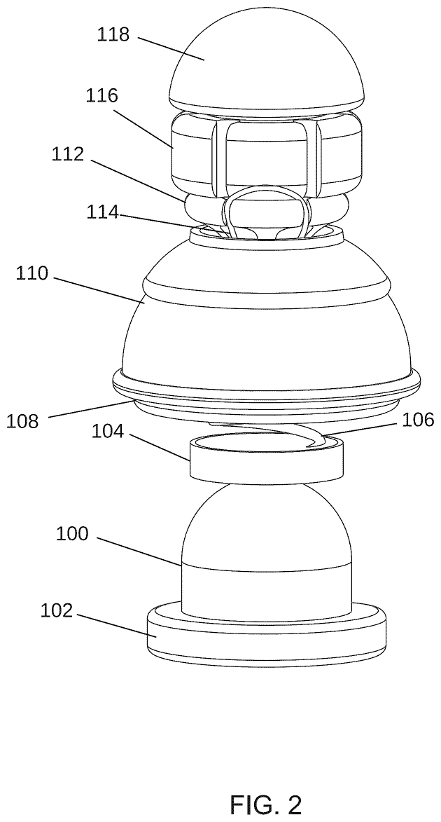

FIG. 2 shows the device in a compressed configuration, where the spring 106 is compressed, thereby reducing the distance between the first skin-contacting assembly 108 and the second skin-contacting assembly 100. This configuration demonstrates the dynamic tensioning capability of the spring 106, which adapts to user movement or anatomical variations. The retention mechanism 112 remains in the same position, and the elastic cords 114 maintain their adjustable properties, allowing fine-tuning of the orientation of the first skin-contacting assembly 108 even as the spring 106 changes its state.

FIG. 3 illustrates the same device configuration as FIG. 2 but with the outer gripping element 110 of the first skin-contacting assembly 108 inverted and raised to reveal the inner gripping element 111. This adjustment facilitates the placement or removal of the foreskin and improves the securement during use. The inner gripping element 111 may feature a ridge or textured surface to assist in aligning and holding the foreskin securely. Both the inner and outer gripping elements may be made of flexible materials and are designed to accommodate variations in skin length and tautness.

Although FIG. 1, FIG. 2, and FIG. 3 show a single spring 106 in the tensioning assembly, alternative configurations are possible. For example, the spring 106 may be replaced or supplemented with a second spring of different stiffness or length to achieve customized tensioning. The retention mechanism 112, while shown in conjunction with a spring 106, could alternatively be coupled to a fixed spacer element for a simplified configuration. The modular coupling elements, not visible in these figures but integral to the assembly, ensure that all components can be easily interchanged or replaced, including variations in the first and second skin-contacting assemblies or tensioning mechanisms.

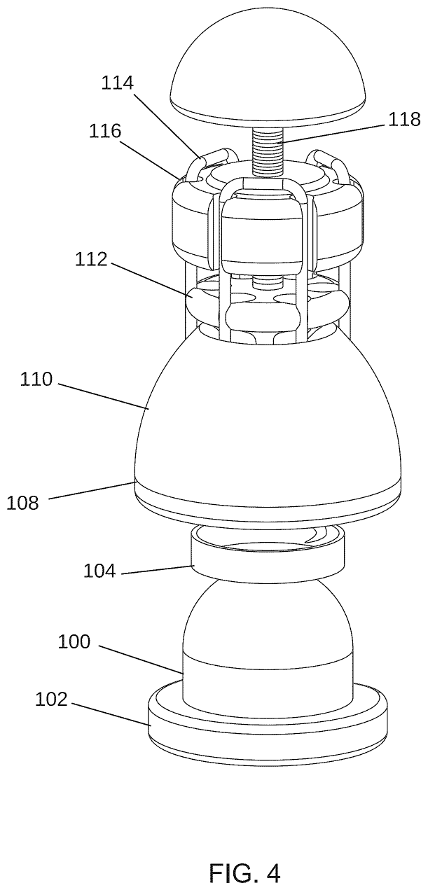

FIG. 4 illustrates the modular foreskin restoration device in a configuration where the elastic cords have been engaged with the adjuster knob, demonstrating how tension can be applied and adjusted. This figure provides a detailed view of the retention mechanism and its interaction with the elastic cords and threaded rod.

The first skin-contacting assembly 108 is visible below the retention mechanism 112. The outer gripping element 110 is in its standard position, configured to securely hold the foreskin. The retention mechanism 112 includes a body portion with circumferential grooves through which the elastic cords 114 are threaded. Three elastic cords 114 are visible in this figure, looped through the grooves of the retention mechanism 112 and extending to the adjuster knob 116 above. The elastic cords 114 are flexible and adjustable in length, allowing the user to control the tension applied to the foreskin by threading or rethreading the cords through the retention mechanism 112 and adjuster knob 116. The cords may be made of elastomeric materials such as medical-grade silicone to ensure durability and biocompatibility.

The adjuster knob 116 is positioned above the retention mechanism 112 and includes circumferential grooves for securing the elastic cords 114. The knob 116 is connected to a threaded rod 118 that extends downward through the central threaded opening of the retention mechanism 112. In this figure, the threaded rod 118 has been rotated, causing it and the adjuster knob 116 to be raised upwards relative to the retention mechanism 112 and the first skin-contacting assembly 108. This upward movement of the adjuster knob 116 increases the tension in the elastic cords 114, thereby modifying the orientation and force applied by the first skin-contacting assembly 108.

The spring 106, which is positioned below the first skin-contacting assembly 108 and extends down to the second skin-contacting assembly 100, remains uncompressed in this configuration. This illustrates that the adjustment of tension via the elastic cords 114 is independent of the compression state of the spring 106, enabling dual mechanisms for tension control: axial force provided by the spring 106 and angular or localized tension adjustments via the elastic cords 114.

The second skin-contacting assembly 100, located at the bottom of the device, remains unchanged in this figure, with its dome-shaped surface configured to engage the glans of the user. Although not visible in this view, the modular coupling elements at the ends of the spring 106 allow for interchangeability and customization of the device, including the use of springs with varying stiffnesses or spacers for fixed-length configurations.

This figure also demonstrates how the adjuster knob 116 and threaded rod 118 enable precise tension adjustments. By rotating the threaded rod 118, the user can raise or lower the adjuster knob 116, thereby altering the tension in the elastic cords 114 and the orientation of the first skin-contacting assembly 108. In some embodiments, the threaded rod 118 may be replaced with a fixed-length spacer for a simplified, non-adjustable configuration.

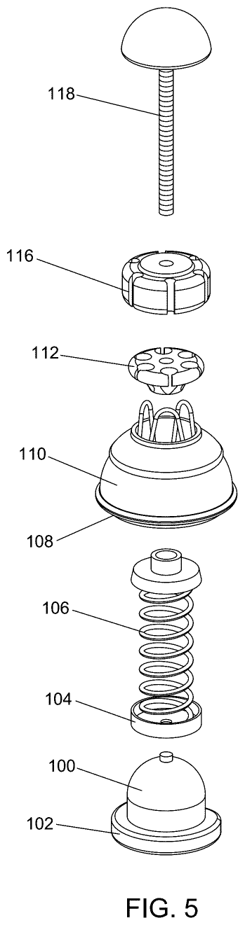

FIG. 5 illustrates an exploded view of the modular foreskin restoration device, showing all individual components in their unassembled state. This view highlights the modularity and interchangeability of the components, emphasizing the flexibility of the design and the connections between each part.

At the top of the figure is the adjuster knob 116, which includes a domed cap with circumferential grooves. The adjuster knob 116 interacts with the threaded rod 118, allowing for fine-tuning of the tension applied to the elastic cords. The adjuster knob 116 may be constructed from materials such as plastic or lightweight metal for durability and ease of adjustment. Below the adjuster knob 116 is the threaded rod 118, which is shown as a single elongated structure with external threading. The threaded rod 118 engages with the central threaded opening of the retention mechanism, providing axial adjustability. In some configurations, this component may be replaced with a fixed spacer element for simplified operation.

Beneath the threaded rod 118 is the retention mechanism 112, consisting of a circular body with openings distributed around its outer circumference. These openings are designed for threading the elastic cords 114, which adjust the orientation and tension of the first skin-contacting assembly 108. The retention mechanism 112 also includes a central threaded opening for engagement with the threaded rod 118. This component may be made from a rigid material such as polycarbonate or reinforced plastic to withstand repeated adjustments.

Below the retention mechanism 112 is the first skin-contacting assembly 108, which includes an outer gripping element 110 and an inner gripping element (not visible in this view). The outer gripping element 110 is shaped to secure the foreskin during use and can be inverted to reveal the inner gripping element, as shown in FIG. 3. Both gripping elements may be made from flexible, biocompatible materials such as silicone to ensure user comfort and effective gripping.

The spring 106 is shown positioned between the first skin-contacting assembly 108 and the second skin-contacting assembly 100, with a first modular coupling element at its upper end and a second modular coupling element at its lower end. The spring 106 is depicted as a helical compression spring, but alternative configurations such as dual springs or springs with varying stiffness and lengths may be employed. The spring 106 provides dynamic tension along the axis of the device and is adjustable through the modular coupling elements.

At the base of the figure is the second skin-contacting assembly 100, which features a dome-like structure for engaging with the glans. This component includes a contact surface that may be concave or incorporate an air pad to provide additional cushioning and comfort. The second skin-contacting assembly 100 connects securely to the lower modular coupling element of the spring 106 and may be constructed from biocompatible materials such as medical-grade silicone or thermoplastic elastomers.

While the figures provide a detailed view of the modular foreskin restoration device in various configurations, several components and potential variations are not shown but remain integral to the flexibility and adaptability of the design.

One such component is the spacer element, which may be used in place of the threaded rod and adjuster knob assembly for configurations where a fixed separation distance between the retention mechanism and the first skin-contacting assembly is preferred. The spacer element is a rigid structure that can be inserted between the modular coupling points of the device. In some implementations, the spacer may be made of lightweight materials such as bioplastics, aluminum, or reinforced polymers to ensure durability while maintaining a minimal profile. Spacers may be provided in various lengths to accommodate users with different anatomical requirements or preferences for tension levels.

Alternative spring elements can replace or supplement the primary spring 106 shown in the figures. These alternative springs may include dual-spring arrangements or springs with varying diameters, lengths, or stiffness levels. For example, a heavy-duty spring may be used in conjunction with a standard spring to increase the overall tensioning force and stability. These alternative springs enhance the versatility of the device by enabling users to adjust the tensioning force to suit their stage of restoration or desired level of tension.

The elastic cords depicted in the figures are shown threaded through the grooves of the retention mechanism and adjuster knob. However, the disclosure contemplates variations in the materials and configurations of these cords. For example, the cords may include additional adjustment mechanisms such as tension locks, clasps, or sliding knots to provide more precise control over their length and tension. These cords may also be color-coded or provided with markings to assist users in achieving consistent adjustments.

Other potential attachment mechanisms for the modular coupling means could be used. These include snap-fit connectors, bayonet connectors, friction fits, and adhesive interfaces, which may be used in place of the threaded connectors shown in the figures. Such alternative coupling mechanisms offer additional assembly and disassembly options, further enhancing the usability of the device.

Customizable gripping elements for the first skin-contacting assembly are also contemplated. While the figures depict a standard inner and outer gripping configuration, variations such as textured or ribbed surfaces, additional alignment ridges, and replaceable gripping components to suit different skin types and user preferences are also possible. These variations improve the adaptability of the device for individuals with varying degrees of foreskin length or tautness.

Finally, the disclosure contemplates additional features for the second skin-contacting assembly, such as an integrated air pad or cushioning structure adjacent to the glans-contacting surface. This unillustrated feature may provide increased comfort during extended wear and reduce pressure points on the glans. The second skin-contacting assembly may also include customizable configurations, such as interchangeable contact surfaces or padding materials.

### CONCLUSION

Unless otherwise defined, all terms (including technical terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

The disclosed embodiments are illustrative, not restrictive. While specific configurations of the device of the invention have been described in a specific manner referring to the illustrated embodiments, it is understood that the present invention can be applied to a wide variety of solutions which fit within the scope and spirit of the claims. There are many alternative ways of implementing the invention.

It is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. A modular foreskin restoration device, comprising:
a first skin-contacting assembly configured to secure a portion of the foreskin, the first skin-contacting assembly comprising inner and outer gripping elements and including a first coupling means;
a second skin-contacting assembly positioned in spaced relation to the first skin-contacting assembly, the second skin-contacting assembly comprising a contact surface configured to engage with the glans of a user;
a tensioning assembly disposed between the first skin-contacting assembly and the second skin-contacting assembly, the tensioning assembly comprising:
(i) a first modular coupling element positioned at one end of the tensioning assembly;
(ii) a second modular coupling element positioned at an opposite end of the tensioning assembly; and
(iii) at least one spring element positioned between the first and second modular coupling elements and operable to provide an adjustable force along an axis between the first and second skin-contacting assemblies;
a retention mechanism disposed above the first skin-contacting assembly, the retention mechanism comprising:
a body portion with a plurality of openings positioned about its outer circumference for threading elastic cords; and
a central opening for aligning with the axis;
a modular coupling means operable to detachably connect the first skin-contacting assembly, the second skin-contacting assembly, the tensioning assembly, and the retention mechanism, wherein the modular coupling means permits interchangeability of components within the device;
wherein the elastic cords are threaded through the openings in the retention mechanism and the top of the first skin-contacting assembly and are adjustable in length to modify the tension applied to the foreskin and the orientation of the first skin-contacting assembly with respect to the axis; and
wherein the first and second skin-contacting assemblies are each configured to allow relative angular and positional adjustment with respect to the axis.

2. The foreskin restoration device of claim 1, wherein the first skin-contacting assembly comprises an inner and an outer gripping element, the inner and outer gripping elements being concentrically arranged to secure the foreskin between them.

3. The foreskin restoration device of claim 2, wherein the inner and outer gripping elements are constructed from a flexible material.

4. The foreskin restoration device of claim 1, wherein the tensioning assembly further comprises a second spring element, the second spring element being combinable with the at least one spring element to provide an increased adjustable force.

5. The foreskin restoration device of claim 4, wherein the at least one spring element is a first spring having a first diameter and a first stiffness, and the second spring element has a second diameter and a second stiffness, the second stiffness being greater than the first stiffness.

6. The foreskin restoration device of claim 1, wherein the retention mechanism comprises a central threaded opening and is operable to adjust the axial position of the retention mechanism relative to the first skin-contacting assembly.

7. The foreskin restoration device of claim 6, further comprising a threaded rod inserted through the central threaded opening, the threaded rod being operable to control the distance between the retention mechanism and the first skin-contacting assembly.

8. The foreskin restoration device of claim 6, wherein the retention mechanism is configured to allow substitution of the threaded rod with a spacer element of fixed length.

9. The foreskin restoration device of claim 1, wherein the modular coupling means comprises a set of interlocking fasteners selected from the group consisting of threaded connectors, snap-fit connectors, and bayonet connectors.

10. The foreskin restoration device of claim 1, wherein the retention mechanism comprises a plurality of openings distributed radially about its outer circumference, the openings being configured to allow elastic cords to be threaded through.

11. The foreskin restoration device of claim 10, wherein the elastic cords are adjustable in length to modify the tension applied to the foreskin and the orientation of the first skin-contacting assembly.

12. The foreskin restoration device of claim 1, wherein the second skin-contacting assembly comprises a concave surface configured to conform to the glans of a user.

13. The foreskin restoration device of claim 1, wherein the modular coupling means permits the substitution of alternative tensioning assemblies, each alternative tensioning assembly comprising a different tensioning mechanism.

14. The foreskin restoration device of claim 1, wherein the housing of the tensioning assembly is configured to accommodate springs of varying lengths to enable customization of the tensioning force.

15. The foreskin restoration device of claim 1, wherein the at least one spring element is a compression spring.

16. The foreskin restoration device of claim 1, wherein the tensioning assembly further comprises a guiding structure configured to maintain alignment of the at least one spring element along the axis.

17. The foreskin restoration device of claim 1, wherein the first skin-contacting assembly further comprises a foreskin alignment ridge positioned to assist in securing the foreskin.

18. The foreskin restoration device of claim 1, wherein the second skin-contacting assembly further comprises an air pad positioned adjacent to the contact surface.

19. The foreskin restoration device of claim 1, wherein the device is configured to be worn discreetly under clothing, with the retention mechanism and the first and second skin-contacting assemblies being compact in size.

20. The foreskin restoration device of claim 1, wherein the first skin-contacting assembly is detachably coupled to the retention mechanism via a Velcro fastener, a friction fit, or an adhesive material, enabling ease of assembly and disassembly.
